(19) United States
(12) Patent Application Publication
(10) Pub. No.: US 2017/0148171 A1
(43) Pub. Date: May 25, 2017

(54) Patient Body Contour Delineation Aid

(71) Applicant: Target Tape Inc., Surrey (CA)

(72) Inventor: Nicholas Drake Seto, Surrey (CA)

(73) Assignee: Target Tape Inc., Surrey (CA)

(21) Appl. No.: 15/356,541

(22) Filed: Nov. 19, 2016

Related U.S. Application Data
(60) Provisional application No. 62/258,682, filed on Nov. 23, 2015.

(51) Int. Cl. G06T 7/00; A61N 5/10; A61B 6/00; A61B 6/03

(52) U.S. Cl. CPC G06T 7/0085; A61B 6/032; A61N 5/1039; A61N 5/1077; A61B 6/5205; G06T 7/0081; G06T 7/0012; G06T 2207/10081

(57) ABSTRACT

A body contouring aid for use in medical scan imaging such as CT simulation scanning, and methods of using the aid are disclosed. The aid includes a reference pattern defined by at least partially radiopaque indicia material applied to a substrate. The substrate is applied to a patent's body in a portion of the body that extends beyond the CT simulator's scan field of view (sFOB) but remain within the simulator's extended field of view (eFOV). A medical scan image generated by a CT simulator show a visualization of both the patient's internal anatomy and reference marks that correlate to the at least partially radiopaque indicia from the body contouring aid. The reference marks are used to create an accurate delineation of that portion of the patient's body that is in the eFOV so that an accurate source-to-surface distance may be determined for accurate radiation treatment.

### TECHNICAL FIELD

The present invention generally relates to apparatus and methods used in radiation therapy. More particularly, the present invention relates to apparatus and methods for creating an improved body contour delineation for computed tomography (CT) simulation needed for external beam radiation therapy.

### BACKGROUND

A patient diagnosed with cancer has several options for treatment. Popular treatments include surgery, chemotherapy and radiation therapy. Radiation therapy, also known as radiotherapy, is therapy using ionizing radiation, generally as part of cancer treatment to control or kill malignant cells. External beam radiation therapy directs a beam of radiation through the skin to the tumor and a small amount of normal surrounding tissue. The present invention relates to external beam radiation therapy.

A computed tomography (CT) simulator is commonly utilized in CT simulation to plan radiation therapy treatment. CT simulation is a process used by the radiation therapy team to determine the exact location, shape, and size of the tumor to be treated. CT simulation also provides information about the patient's external and internal anatomy. A CT simulator can produce images showing a transverse cross section of the patient's body. These cross sectional images, or slices, can have an axial thickness ranging from 0.20 cm to 0.50 cm wide, and show the patient's body contour delineation, internal structures and tumor. The planning target volume (PTV) includes the tumor and is the area of interest that needs to be treated. The depth of the PTV is known as the source-to-surface distance (SSD), which measures the distance of the PTV to the surface of the patient. The SSD is one variable amongst others that are required to determine an appropriate, accurate treatment plant.

Popular CT scanners a clinical facility may use include the GE LightSpeed RT, Siemens SOMATOM Definition AS Open RT Pro, Philips Brilliance CT Big Bore and Toshiba Aquilion LB. The GE LightSpeed RT has a scan field of view (sFOV) diameter of 50 cm; however, by employing extrapolation methods and image reconstruction algorithms, the GE LightSpeed RT (and other CT simulators) can reconstruct CT images at an extended field of view (eFOV) with a diameter of 65 cm. The Siemens SOMATOM Definition AS Open RT Pro has a maximum sFOV of 50 cm and eFOV of 80 cm. The Philips Brilliance CT Big Bore has a maximum sFOV of 60 cm and eFOV of 70 cm. Lastly, the Toshiba Aquilion LB has a sFOV of 70 cm and eFOV of 85 cm.

For larger sized patients, the sFOV may not be large enough to produce a full cross sectional image of the patient's torso and other body areas. In some instances, a patient that is of smaller size may be positioned outside the sFOV due to positioning reasons or by accidental misposition. The body areas that fall outside the sFOV and utilizes the eFOV reconstruction, do not appear clearly on the resulting images. The eFOV reconstruction introduces artifacts and body contour distortion in the CT images due to truncated projection data collect by the scanner in the eFOV region. This results in an image that does not have an accurate body contour delineation (i.e. distortion) and/or accurate CT numbers. Some Siemens CT scanners/simulators contain an HDFOV, which is similar to an eFOV because it also contains body contour distortion. Referring to eFOV will herein include the HDFOV.

The radiotherapy team heavily relies on the patient's body contour to determine the SSD, which is an important variable used to calculate the treatment dosage. For patients with larger habitus, the body contour within the eFOV becomes distorted in the CT images.

Techniques to solve this problem include the radiotherapy team making manual adjustments to draw an estimated outline of where they think the body contour is. Treatment planning software may also include features that outline internal and external contours. Software can help make manual contour adjustment more efficient, but the imperfect data gathered from the eFOV does not allow the software to make an accurate body contour estimation. This estimated, manually adjusted body contour is often inaccurate and results in the radiation dosage being inappropriately high or low. A dosage that is too low may not be enough to efficiently treat the disease and a dosage that is too high may unnecessarily target and damage healthy body tissues, i.e. critical structures.

Another solution the radiotherapy team may employ is fusing multiple CT simulator images. The patient is placed on an extreme side of the CT simulator bed, for example, on the left side. This results in the patient's right side of the body being within the sFOV and producing a clear image on the right side. The patient is then shifted to the right side of the CT simulator bed and the resulting scan produces a clear image of the patient's left side. These two scans are then fused so that the clear left and right side of the image are joined to create one image with a more accurate body contour. This solution is time consuming and exposes the patient to excessive radiation exposure. It can also be inaccurate due to patient movement.

Other prior art solutions include apparatus and methods of improving medical imaging scans. None of the prior art addresses the problems and solutions of CT simulator body contour distortion.

It is an object of the present invention to overcome one or more of the above described drawbacks and/or disadvantages of the prior art apparatus and methods.

### SUMMARY OF THE INVENTION

The apparatus and method of the present and illustrated invention is based on a device that defines apparatus and methods of creating a body contour delineation within a CT simulator's eFOV image for radiation therapy treatment. The aid is applied to the body of the patient undergoing a CT simulator scan during CT simulation. The aid will create an improved visual body contour of the body areas that extend beyond the CT simulator's sFOV, but remain within the eFOV. The body contour of the areas in the eFOV may appear distorted and/or unclear in the CT images; however, the materials used in this apparatus are of a density ideal enough to appear as distinct marks without distortion, or minimal distortion. Upon application, the CT simulator images show a visualization of both the internal anatomy and the at least partially radiopaque indicia from the aid itself, herein referred to as the reference marks—the word radiopaque meaning herein a material that would appear on a CT simulator's image. The reference marks will be used to create an accurate outline of the patient's body contour and can then be used to calculate SSD.

The body contouring aid must satisfy four primary constraints to achieve its purpose of producing a clear body contour in a CT simulator image. These four constraints are: at least partially radiopaque pattern, conformability to the patient's body contour (i.e. skin), attachment to the skin, and covering a region of interest (ROI) that falls within the eFOV. Once these four constraints are met, additional features and benefits may be included to produce preferred embodiments that are user friendly and economical.

There are several possible embodiments for the aid. In one embodiment, the aid is a flexible substrate with top and bottoms sides in strip form that is housed in a dispensing structure as a roll and can be attached to a ROI. For purposes herein, a ROI is an area of the body that could fall within the eFOV and if it does, its resulting body contour appears distorted in a medical image scan. For example, a ROI can be, but not limited to, the shoulders, breast, torso and pelvis. The aid contains a radiopaque reference pattern in a plurality of parallel lines that will appear on the CT simulator cross sectional image as a series of dots. The density of the radiopaque material used in this invention is high enough so that the aid will appear distinctly in the distorted areas of the CT image and produce a reasonably accurate body contour.

In accordance with another aspect of the invention, a preferred method for use of the apparatus comprises the following summarized steps:
(i) adhesively and conformably attaching a second side of a flexible substrate to a ROI on a subject's skin, that is, the patient's skin, in the appropriate direction, wherein the substrate includes a plurality of at least a partially radiopaque lines as a reference pattern and spaced relative to each other on a first side of the substrate opposite the second side;
performing a CT simulator scan; and
connecting the reference marks that appear on the CT simulator scan image using treatment planning software currently utilized by the radiation therapy team.

Accordingly, several objects and advantages of the invention are: efficiently dispense and conformably attach an apparatus to a ROI on a patient's skin in a user friendly manner; to provide accurate body contour in the form of reference marks in a CT simulator's eFOV; and to reduce dependence on interpolations, extrapolation and image reconstruction methods of CT simulators. Other objects and advantages of the present invention, and/or of the currently preferred embodiments thereof, will become more readily apparent in view of the following detailed description of the currently preferred embodiments, other embodiments, and accompanying drawings.

### BRIEF DESCRIPTION OF THE DRAWINGS

The invention will be better understood and its numerous objects and advantages will be apparent by reference to the following detailed description of the invention when taken in conjunction with the following drawings.

FIG. 1 is a top plan view of length of a flexible strip comprising the body contour aid according to the present invention;

FIG. 2 is a cross-sectional view of the body contour aid of FIG. 1 taken along the line 2-2 of FIG. 1, and in which the thicknesses of the various layers is exaggerated in order to better illustrate the invention;

FIG. 3 is a bottom plan view of the body contour aid shown in FIG. 1;

FIG. 4 is a bottom view of the body contour aid according to the present invention with the adhesive release strip removed to illustrate an alternative embodiment of the adhesive layer;

FIG. 5 is a perspective view of an elongate strip of the body contour aid according to the present invention wound around a core to form a roll;

FIG. 6 is a transparent perspective view of the roll of body contour aid shown in FIG. 5 contained in a dispensing system and wherein the portions of the roll that are in the interior of the dispensing system are shown in phantom lines;

FIG. 7 is a schematic and not to scale front elevation view of a CT simulator and a patient lying supine on the CT bed associated with the simulator;

FIG. 8 is a schematic front elevation view of a patient in a supine position on a CT simulator bed and illustrating the portions of the patient's body that cross the eFOV border line for the CT simulator;

FIG. 9 is a schematic front elevation view similar to the view of FIG. 8 and showing the patient in a supine position on a CT simulator bed with a ruler placed above the patient to illustrate the eFOV for the patient;

FIG. 10 is a perspective side view of a patient in a supine position on a CT simulator bed with the bed ready to be moved into the CT simulator, with strips of the body contour aid according to the present invention applied to the sides of the patient's pelvis and torso, and showing a medical professional kneeling at eye level at the bottom of patient-foot-end of the bed;

FIG. 11 is a schematic and perspective view of a cylindrical phantom that represents a portion of a human body with a strip of body contour aid according to the present invention attached to the phantom;

FIG. 12 is a schematic radiographic image slice of a CT simulation scan of the phantom of FIG. 11, wherein the image slice is taken along the line 12-12 of FIG. 11;

FIG. 13 is a radiographic image slice of a CT simulation scan, showing a cross section of strips of the body contour aid attached to the surface of a phantom cylinder as shown in FIGS. 11 and 12, wherein the phantom cylinder is placed partially into the eFOV;

FIG. 14 is a radiographic image slice of a CT simulation scan of pelvic portion of a human body upon which a body contour aid according to the present invention has been applied, showing the cross section of strips of the body contour aid placed partially into the eFOV;

FIG. 15 is a top plan view of length of a flexible strip comprising a second embodiment of the body contour aid according to the present invention;

FIG. 16 is a top view of multiple strips of the body contour aid shown in FIG. 15 laid side-by-side, with the resulting cross-sectional CT scan juxtaposed beneath the strips;

FIG. 17 is a schematic illustration of an alternative embodiment of the body contour aid according to the invention and more particularly, a wearable aid;

FIG. 18 is a schematic and perspective view of a cylindrical phantom that represents a portion of a human body that is wearing the body contour aid according to the present invention shown in FIG. 17.

### DETAILED DESCRIPTION OF ILLUSTRATED EMBODIMENTS

The invention will now be described in with reference to the drawings. The embodiments disclosed herein are to be considered exemplary of the principles of the present invention and various modifications will be apparent to those skilled in the art based on the teachings herein without departing from the scope or spirit of the invention described in the specification or in the claims.

The term “reference pattern” is used herein to mean without limitation a plurality of spaced lines or other markings, which may be used, for example, to create a series of reference marks, which will appear as a series of dots or dashes, that can be connected on the CT simulator medical scan image, or other imaging modality medical scan image, and create a body contour in a treatment planning software used for radiation therapy treatment.

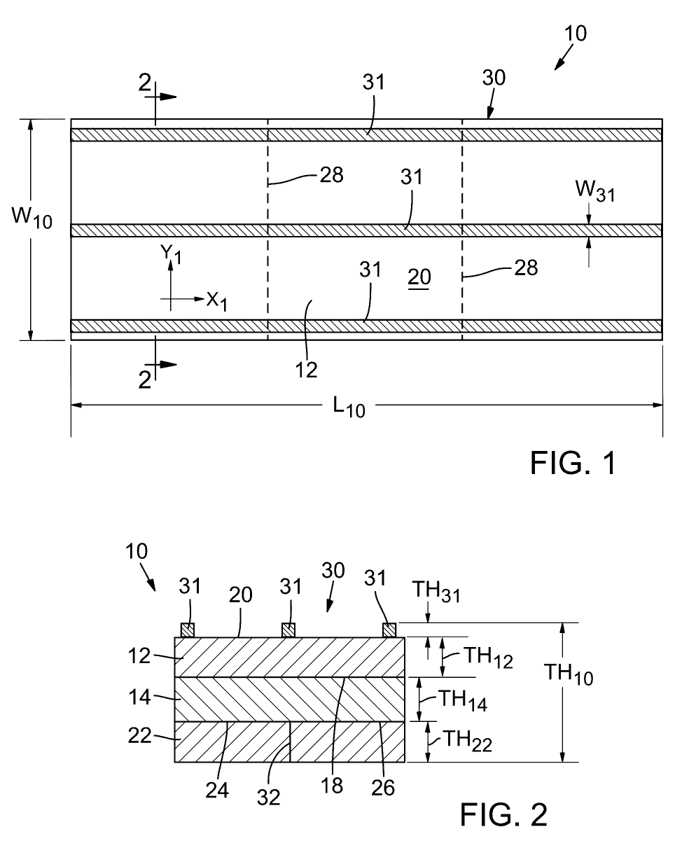

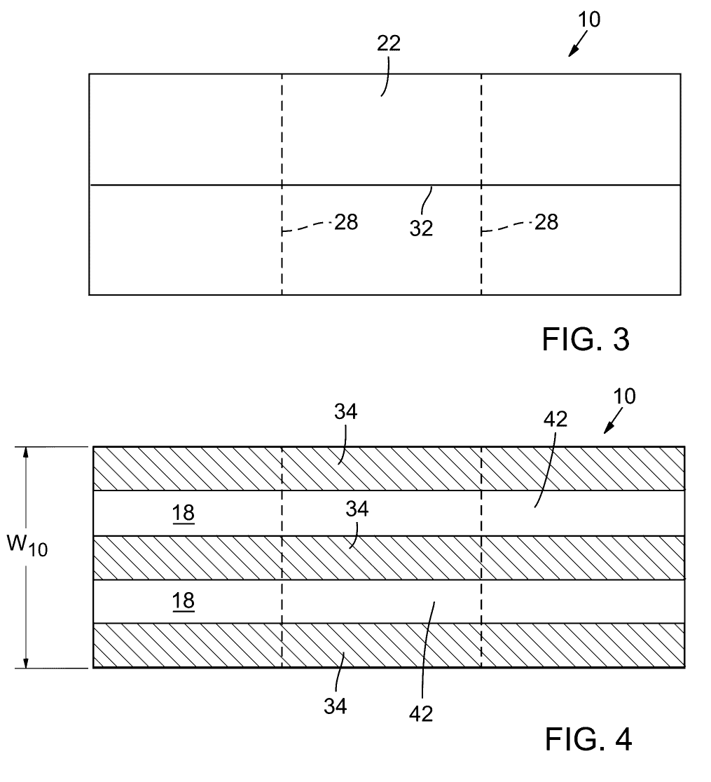

In FIGS. 1, 2 and 3, an adhesive body contour delineation aid is indicated generally by the reference numeral 10. The body contour delineation aid 10 is defined by a strip that includes a substrate or label layer 12 that has an upper surface 20 and an opposite, lower surface 18. A pressure sensitive adhesive layer 14 is attached or applied to the lower surface 18 of the substrate layer 12 and as seen in FIG. 2, the adhesive layer 14 is coextensive in dimensions with the substrate layer. The aid 10 includes an adhesive-protective release liner 22 that is releasably attached at an upper side 24 thereof to the underside 26 of the adhesive layer 14. A “kiss cut” 32 is formed in the release liner 22 along the length of the aid 10 to, as detailed below, facilitate removal of the release liner from the adhesive layer.

As a naming convention, for purposes herein the “first” or “upper” side of the substrate layer 12 is exposed when the aid 10 is in use and hosts a reference pattern 30 that defined by plural elongate strips 31 of material that is at least partially radiopaque to CT simulator radiation or other medical imaging modalities. This “first” or “upper” surface 20 faces away from the patient when the body contouring aid 10 is in place on the patient. The “second” or “lower” surface 18 is the surface that is opposite surface 20. That is, lower surface 18 is the surface onto which the adhesive layer 14 is applied and in use with a patient, the lower surface 18 is the surface of aid 10 facing the patient's body.

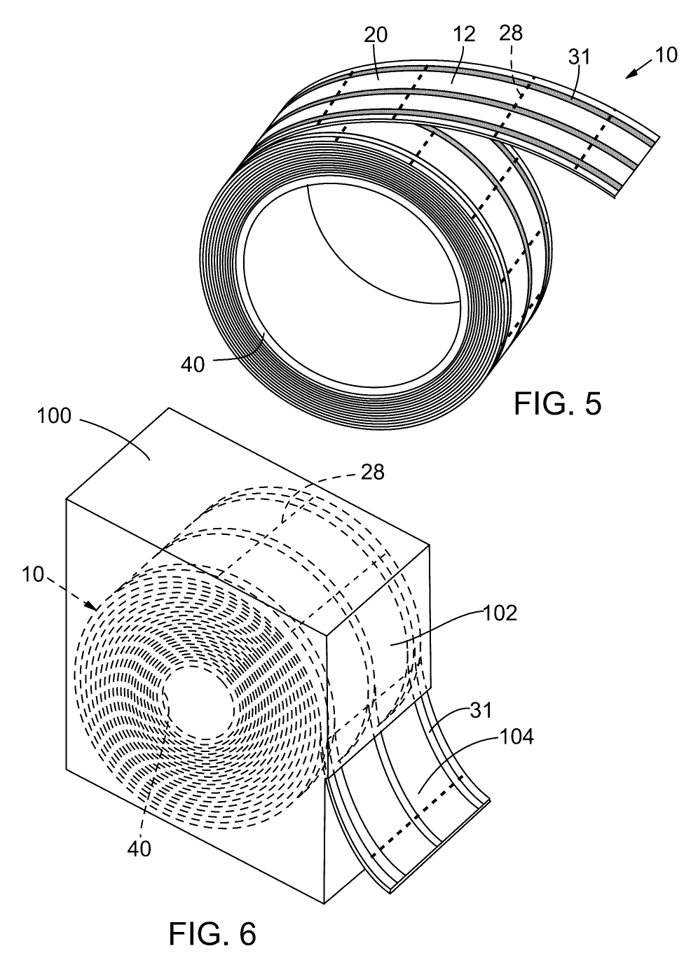

In the embodiment in FIGS. 1 through 3, the contour aid 10 is a substantially rectangular strip. The contour aid 10 generally defines, but is not limited to, a width W₁₀ that is preferably about 2¼ inches and a length L₁₀ that can vary from about 12 inches to several hundred feet or more with the aid 10 in a rolled format as shown in FIG. 5. The aid 10 also defines a thickness TH₁₀ within the range of about 5.0 mils to about 12.0 mils. However, as will be appreciated by those of ordinary skill in the art, other contour aid size and thickness may also be utilized according to the desired application area, such as, for example, the part of the body to which the aid is to be applied and the size of the ROI that is within the eFOV.

Both the substrate layer 12 and the adhesive layer 14 are sufficiently flexible to conformably place the aid 10 atop an underlying curvilinear and irregular surface topography, e.g., the skin surface of a patient, substantially without wrinkling, folding, buckling, distorting or gapping between the aid 10 and the skin surface. The adhesive 14 is sufficiently distributed over the lower side or lower surface 18 of the substrate layer 12 in a sufficient amount to sufficiently hold the aid 10 to the contours of the skin surface, e.g., substantially without wrinkling or leaving gaps there between. Minimizing wrinkling or gaps between the aid 10 and skin surface is important in producing the most accurate body contour in the CT simulator image.

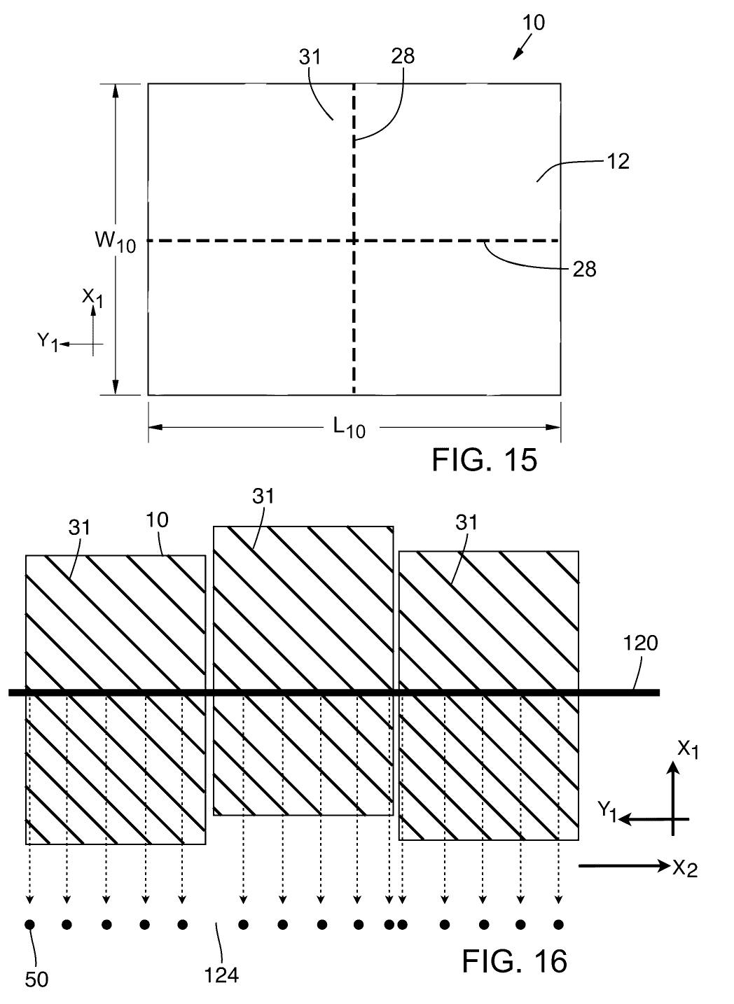

As noted above, in some embodiments the substrate layer 12 further includes a reference pattern 30 defined by plural strips 31 of a material that is at least partially radiopaque. Preferably, the reference pattern 30 and strips 31 are located on the upper surface 20 of the substrate layer 12 relative to the adhesive layer 14, as shown in FIGS. 1 and 2. The reference pattern 30 is preferably defined by a plurality of long, parallel strips 31 that are spaced consistently apart on the surface 20 of substrate 12. Preferred spacing between these parallel strips 31 is about 1.0 inches, but the between-strip spacing may range from about 0.5 to about 2.0 inches. The direction in which the strips 31 of the reference pattern 30 run is parallel to the length or longitudinal axis of the aid 10 and is referred to herein as the “X₁” direction, as illustrated with the directional grid in FIG. 1. The direction that is perpendicular to the direction of the lines of the reference pattern 30 in FIG. 1 is referred to herein as the “Y₁” direction. The “X₁” and “Y₁” directions shown in FIG. 1 are for reference only; they do not appear on the aid itself. As seen in FIG. 1, the reference pattern 30 visually contrasts with the substrate 12 and/or the patient's skin for viewing by the user. In FIG. 1, the reference pattern 30 extends approximately from one side of the substrate layer 12 to the other across the Y₁ direction. In other words, the three strips 31 shown in FIG. 1 are evenly spaced across the Y₁ direction with the two laterally outermost strips located near the side edges of the substrate 12. In some exemplary embodiments as shown in FIGS. 15 and 16, discussed below, the reference pattern 30 contains a plurality of parallel strips 31 that are oriented on substrate 12 between 0 and 45 degrees relative to the X₁ direction.

As noted, the reference pattern 30 is defined by plural strips 31 that comprise an at least partially radiopaque material, in order to be visible in a radiographic image or scan, such as a CT simulator. In one such exemplary embodiment, the strips 31 may be formed of a radiopaque ink such as, for example, but not limited to, tungsten non-lead radiopaque ink. One example of a suitable radiopaque ink used for the reference pattern 30 is tungsten non-lead radiopaque ink sold by Creative Materials, Inc. of Ayer, Mass. In some such embodiments, the ink is printed onto the substrate layer 12. The strips 31 are not limited to a radiopaque ink, but can also be a copper, lead or other radiopaque metal or material that is printed, adhered or suspended into the substrate 12.

Generally, the strips 31 of reference pattern 30 define a strip width W₃₁ along a substantial portion of each strip of between about 1/16^{th} and ¼ inch. The width W₃₁ of the strips 31 of reference pattern 30 is selected so as to be visible to the user and also on the radiographic image or medical scan image, yet reduce blocking or obscuring the visible surface of the skin or underlying tissue in the image or scan. With a width W₃₁ of less than 1/16^{th} inch, the strips 31 may not be sufficiently visible on the scan image. A width W₃₁ of greater than about ¼ inch could cause excessive artifact of reference marks 50 within the eFOV area. The thickness TH₃₁ of the reference strips 31 needs to be an appropriate thickness so it is suitably visible on the CT image scan. Thickness TH₃₁ can range from about 0.3 mils to about 0.6 mils; however, thicknesses of the strips can range based on the radiopaque material's density. Those of ordinary skill in the art should understand that the width of the lines can be selected as appropriate for the intended application.

The aid 10 illustrated in FIG. 1 comprises three strips 31 to form the reference pattern 30. It will be appreciated, however, that more or less strips 31 may be applied to the substrate 12 to define the reference pattern 30. For example, an aid 10 may be formed with a single strip 31 or with 2 strips 31, or as illustrated, with three strips. Of course, more than three strips 31 may be used as well.

In the illustrated and preferred embodiment, the substrate layer 12 is a polyolefin (“PO”) material, preferably transparent. However, as should be recognized by those of ordinary skill in the pertinent art based on the teachings herein, the substrate layer 12 may be formed of any material capable of performing the functions of the substrate layer. That is, and as described further herein, the substrate layer 12 is conformal to the skin contour of the patient without unreasonable gaps, and is rigid enough for the user to handle without folding or wrinkling upon itself. In some such embodiments, the thickness TH₁₂ of a PO substrate layer 12 is greater than or equal to about 2.0 mils but less than or equal to about 5.0 mils. It should be appreciated that other capable materials of the substrate layer 12 could be greater or less than said thicknesses TH₁₂. A transparent quality allows the radiation therapy team to visually see other markings on the body. Other possible substrate materials include, but are not limited to, polyethylene (“PE”), polyurethane (“PU”) or polyethylene terephthalate (“PET”).

In the illustrated embodiment of FIGS. 1 through 3, the contour aid 10 contains a plurality of perforations 28 that are parallel to the Y₁ direction. The perforations 28 extend completely through all layers of the aid 10, including the strips 31 of reference pattern 30, substrate 12, adhesive 14 and release liner 22, and the function of the perforations 28 is to allow the aid 10 to be torn at the perforations in order to generate individual lengths of aid 10 in a usable, desired length. The number of perforations 28 in any one line of perforations is preferably about 12 per inch, but a range of about 6 to 24 perforations per inch can be used in some embodiments. Twelve perforations 28 per inch prevents the aid 10 from unintentionally tearing under minimal force, but also prevents the need for excessive force that could cause the substrate 12 or release liner 22 to tear at areas off or adjacent to the perforations 28. In a preferred embodiment, the adjacent lines that define perforations 28 are spaced about 2.0 inches apart, which makes measuring lengths easy for a user. In other embodiments, perforations 28 can be spaced between about 1.0 and 12.0 inches apart. A range of perforation spacing can make measuring lengths more user-friendly because different ROIs require different average lengths.

Testing performed by the Applicant has shown that the combination of the above-described features shown in FIGS. 1 through 3, such as the composition, width W₁₀ and thickness TH₁₂ of the substrate 12, contributes to unexpected results. The aid 10 was able to be handled in a user-friendly manner without the aid 10 wrinkling or folding upon itself. The aid 10 could be easily attached to the patient's skin surface and conform to the contour without significant gaps between the aid 10 and the patient's skin. Conforming closely to the skin is important to producing an accurate representation of the body contour. The aid 10 could be easily sized to the ideal length for a given patient and ROI by tearing the aid 10 at the appropriate perforation 28.

The Applicant has also found that the combination of the above width W₃₁ of strips 31, the spacing of parallel lines in the X1 direction, composition and thickness TH₃₀ of the reference pattern 30 also contributed to unexpected results. In some embodiments, the reference pattern 30 produced clear reference marks 50 on the CT simulator medical scan image with minimal artifact in the eFOV, while testing with other embodiments resulted in the reference pattern 30 having significant amounts of artifact in the eFOV area, or the reference marks 50 were not suitably visible in the eFOV area.

The adhesive layer 14, defining a thickness TH₁₄ (FIG. 2) is attached or applied to the lower surface 18 of the substrate layer 12. The adhesive layer 14 is in contact with the patient's skin surface (after release liner 22 is removed and the aid 10 is applied to the skin) and also cooperates with the substrate layer 12 to allow the aid 10 to conform to an underlying curvilinear and irregular surface contour substantially without wrinkling, folding, buckling, distorting or gapping between the aid 10 and the surface, as shown on the right side of the phantom scan image in FIG. 14, as discussed below.

In the illustrated embodiment of FIG. 2, the pressure sensitive adhesive layer 14 is a skin compatible material. However, as should be recognized by those of ordinary skill in the pertinent art based on the teachings herein, the adhesive layer 14 may be formed of any material capable of performing the functions of the substrate layer. That is, and as described further herein, the adhesive layer 14 adheres instantly to the skin when light pressure applied, is conformal to the skin contour of the patient without unreasonable gaps, remains attached to the patient during normal movement required in CT simulation, does not cause unreasonable irritation or pain upon removal from the patient and does not compromise the flexibility of the substrate 12. Upon removal of the aid 10 from the patient, the adhesive layer 14 will be removed along with the substrate so that no adhesive material remains on the patient, and with minimal irritation or pain experienced by the patient. In some such embodiments, the thickness TH₁₄ of the adhesive layer 14 is preferably greater than or equal to 1.0 mils but less than or equal to 3.0 mils.

In the illustrated embodiment, the bond force of the adhesive layer 14 to the lower surface 18 of substrate 12 is greater than the bond force to an underlying surface, such as a patient's skin or medical phantom surface, upon which the aid 10 is adhered using adhesive layer 14. Accordingly, the adhesive layer 14 substantially does not separate from the substrate 12 when the release liner 22 is removed from the aid 10 or when the aid 10 is removed from the patient's skin.

In the illustrated embodiment of FIG. 2, an example of a preferred substrate 12 and adhesive layer 14 combination is FLX062744 Dermaflex PE 370 F Clear PTP H-529 SPEC 50K-8 (1.9-2.1) sold by FLEXcon COMPANY, Inc. of Spencer, Mass. Such a material is easy for a user to handle without the aid 10 wrinkling or folding upon itself; conforms to an underlying curvilinear surface contour substantially without wrinkling, folding, buckling, distorting or gapping between the aid 10 and the surface onto which the aid is adhered; remains attached to the patient during normal movement required in CT simulation; and does not cause unreasonable irritation or pain upon removal from the patient.

As noted above, the release liner 22 is an adhesive-protective layer that is removably adhered to the lower surface 24 of adhesive layer 14. One purpose of the liner 22 is to protect the adhesive layer 14, e.g., until the aid 10 is about to be used. In some embodiments, the liner 22 is a polymer-based material, such as, for example, but not limited to, a polyester liner. In some embodiments, the liner 22 defines a thickness TH₂₂ (FIG. 2) within the range of about 2.0 mils to about 5.0 mils, which has been found to adequately protect the adhesive layer 14. Other suitable materials and thicknesses can also be used. While the liner 22 is intended to remain on the adhesive layer 14 until use, it also can be removed without excessive difficulty. For easier separation of from the adhesive 14, the release liner 22 contains a kiss cut 32 in the X₁ direction.

In some exemplary embodiments, the adhesive layer 14 may, without limitation, be applied entirely to the underside 18 of the substrate layer 12, or alternately in the form of a pattern and/or a matrix. As shown in FIG. 4, for example, the adhesive layer 14 may be comprised of strips distributed along the lower surface 18 of the substrate layer 12 in a series of rows and/or columns 34 extending in the X₁ direction. The adhesive that is used for the adhesive layer 14 may be applied to the lower surface as adhesive dots and/or lines, defining adhesive-free spaces 42 therebetween. Such adhesive-free spaces 42 would not be found along the opposite lateral edges of the aid 10—as may be seen in FIG. 4 the adhesive strips 34 are coextensive with the lateral side edges of the aid 10. With continuing reference to FIG. 4, in one embodiment the width W₁₀ of the aid 10 is about 2¼ inches. The width W₁₀ can be divided into 5 equal rows of 0.45 inches. Three rows would contain strips 34 of adhesive materials, with two of the rows butting coextensively against the opposed lateral edges of the substrate 12 and one row of adhesive extending through the middle. With this arrangement, there are two rows 42 that are adhesive free. As should be appreciated by those of ordinary skill in the pertinent art, the rows/lines and/or dots of adhesive/non-adhesive are spaced as necessary with respect to one another to substantially prevent lifting, gapping or wrinkling between the aid 10 and the skin surface.

The Applicant has also found that the combination of adhesive material and that defines adhesive layer 14 and adhesive free areas 42, such as two rows 42 shown in FIG. 4 and which are about 0.45 inches wide within a W₁₀ of 2¼ inches, exposed on the bottom side 18 of the substrate 12, contributed to unexpected results. Patients with excessive body hair, or body hair that is longer than average, would have a reduced amount of body hair removed upon removal of the aid 10. A reduction in body hair removal, upon removal of the aid, resulted in a more comfortable experience for the patient. The adhesive free areas 42 would reduce the amount of total adhesive in contact with the patient and therefore result in less hair removal.

As discussed above, the contour aid 10 can be used for radiation therapy, specifically during CT simulation when a CT simulator is used. In one exemplary embodiment, the aid 10 is placed on a patient to assist a radiotherapy professional, such as a radiotherapist, dosimetrist or medical physicist in creating an accurate body contour outline in the eFOV. For purposes of example only, an exemplary ROI includes the sides of the hips, sides or top of the stomach, breast and shoulder. These are areas that tend to be within the eFOV due to obesity or patient positioning. The resulting ROI in the eFOV appears to be distorted. The distortion does not provide the means for the radiation therapy team to accurately measure the PTV's SSD, which can affect treatment dosage calculation.

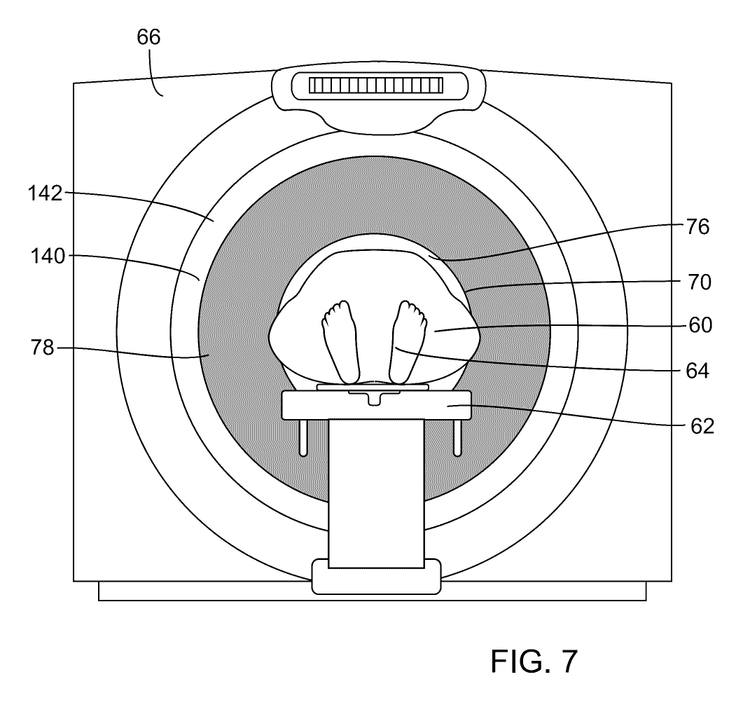

FIG. 7 is a schematic front view of a CT simulator 66 with a patient 60 lying on the CT bed 62 with their feet 64 showing. The schematic of FIG. 7 is based loosely and for illustrative purposes only on a GE LightSpeed RT CT simulator and shows the sFOV 76, which is 50 cm in diameter. Outside the 50 cm sFOV 76 is the eFOV 78 area. Where the eFOV 76 and eFOV 78 meet, is the eFOV border 70. In this example, the eFOV area is between 50 cm-65 cm. Outside the 65 cm eFOV 78 area is the non-image area 140. The bore 142 is the physical circular opening that the patient, bed and/or other objects go through.

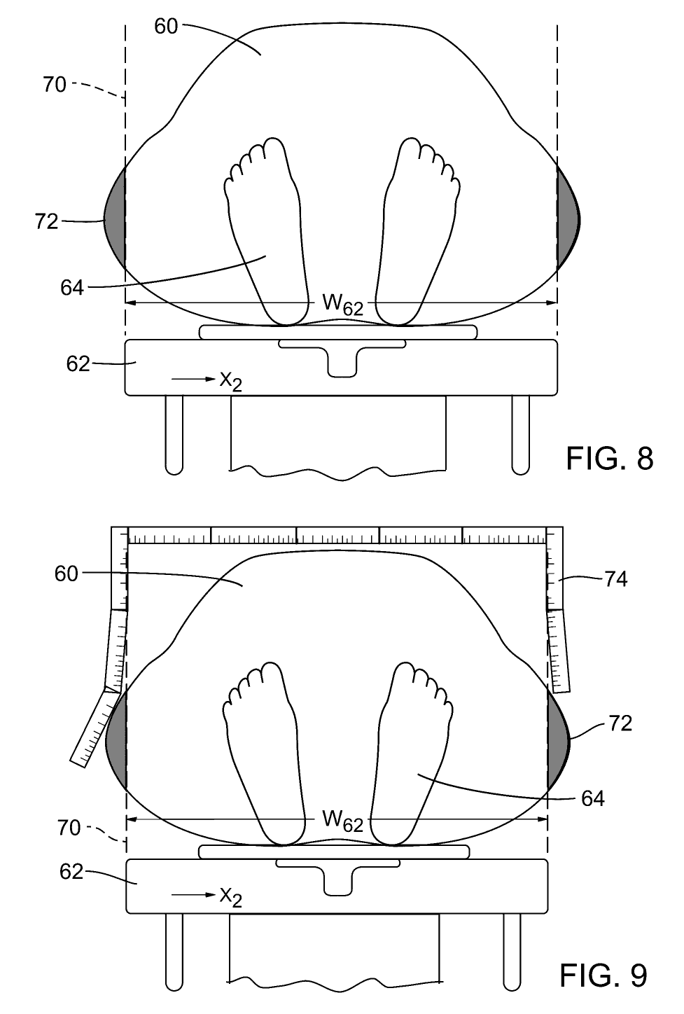

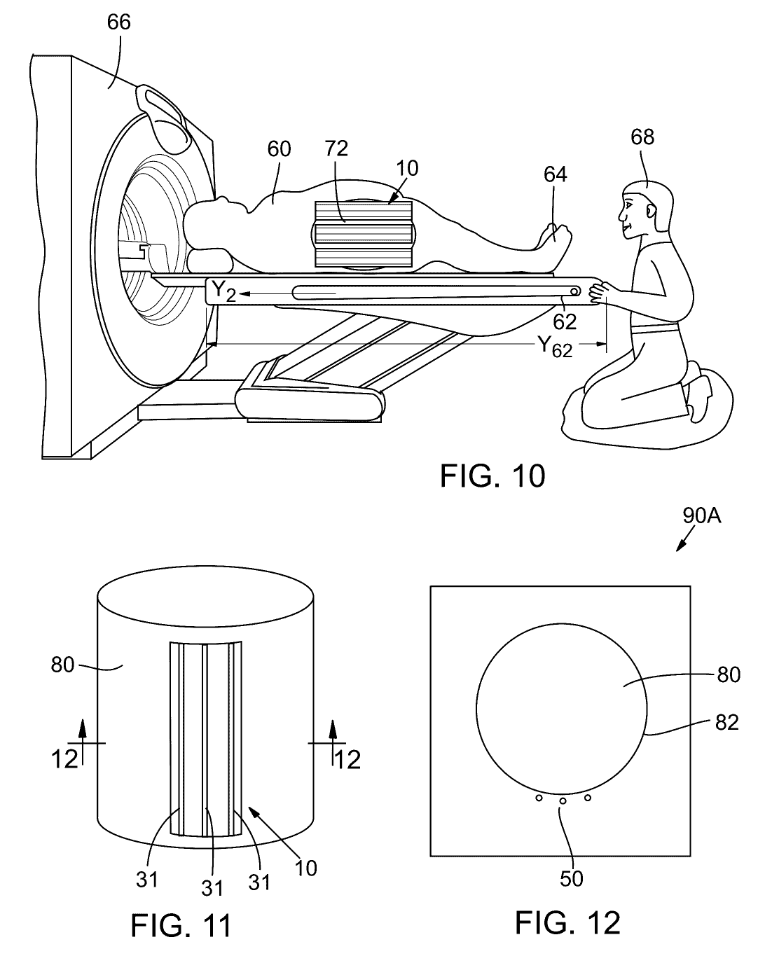

FIG. 8 illustrates a method to qualify a patient 60 needing the aid 10 according to the invention in order to get an accurate SSD. Generally, this occurs when the eFOV is greater than 50 cm in diameter. To determine if a patient 60 will be outside the 50 cm diameter, reference can be made to the CT simulator scan bed and the scan bed width W₆₂. Although different manufacturers provide different bed dimensions, a common bed width W₆₂ is 50 cm, or around 50 cm. The direction in which the bed width W₆₂ run is referred to herein as the “X₂” direction. The direction that is perpendicular to the direction of the bed width W₆₂, i.e. the bed length L₆₂ (shown in FIG. 10), is referred to herein as the “Y₂” direction and the Y₂ direction defines the longitudinal axis of the bed 62. The “X₂” and “Y₂” directions shown in FIGS. 8 and 10 are for reference only; they do not appear on the bed 62 itself. The patient 60 is first placed in the exact position in which they will receive treatment. The patient's feet 64 will be at the bottom of the bed 62, which is the side that is furthest from the CT simulator 66, as seen in the side view of FIG. 10. While the patient 60 is lying down, the radiation therapy professional 68 can crouch down to eye level at the bottom of the bed 62, and look down the Y₂ direction towards the CT scanner 66. For bed widths W₆₂ that are exactly 50 cm, the professional 68 will visualize an eFOV border line 70 that is perpendicular from the edges of the bed width W₆₂ and the floor. Body areas 72 that cross the eFOV border lines 70 will be in the eFOV, i.e., outside of the sFOV, and will be distorted in a CT image. These body areas 72 are the ROIs to which the aid 10 will be applied. For beds 62 that are around 50 cm, either narrower or wider, the professional 68 will make an adjustment to the position of the border lines 70 based on how the bed width W₆₂ compares to the eFOV diameter. For bed widths W₆₂ that are less than 50 cm, the professional 68 will shift the eFOV border lines 70 the appropriate distance to the left and right of the bed width W₆₂. Body areas 72 that cross the adjusted eFOV border lines 70 will be the ROI. For bed widths W₆₂ that are greater than 50 cm, the professional 68 will shift the eFOV border lines 70 to the appropriate distance within the bed width W₆₂. Body areas 72 that cross this adjusted eFOV border lines 70 will be the ROI. One skilled in the art will understand that scanners with an eFOV diameter that is not 50 cm and bed widths W₆₂ that are not 50 cm can make the appropriate adjustments by referencing the bed width W₆₂ to qualify a patient 60 that will need the aid 10.

FIG. 9 illustrates yet another method to assess a patient 60 needing the aid 10 according to the invention. Generally, the eFOV is greater than 50 cm in diameter. To determine if a patient 60 will be outside the 50 cm nominal diameter of the eFOV, a foldable ruler 74 can be used to measure the patient 60. One example of a suitable ruler is the LongLife® 1m Imp./Metric Pocket Ruler. This rigid ruler 74 folds out in 10 cm increments. The ruler 74 can be folded flat from 30 cm-80 cm, which would measure a length of 50 cm. The sections from 0-30 cm and 80-100 cm can be folded down and perpendicular to the 30-80 cm section. This ruler 74 can then be placed over patient 60 to identify body areas 72 that define the ROI, while positioned parallel to the X₂ direction. The ruler 74 is then moved down towards the floor and encompass the patient 60. If the sections of the ruler that extend next to the patient touch the patient 60, then the patient is at least 50 cm in diameter and would require the aid 10 because a body area 72 will be in the eFOV. One skilled in the art will understand that scanners with an eFOV diameter that is 60 cm would adjust the flat portion of the ruler 74 from 20-80 cm instead.

With reference now to FIG. 10, and for purposes of illustration only, multiple strips of the aid 10 may be attached to a large patient 60 on body areas 72 that defines an ROI, i.e. the sides of their stomach, as identified in FIG. 10, and which body areas 72 are in the eFOV for the scanner 66. The multiple strips of aid 10 are oriented so that X₁ is parallel to Y₂. The length and number of strips can vary depending on the size and weight of the patient, body shape, treatment area and the scan area of interest. Each strip that defines an aid 10 will be spaced apart in a distance that is about the same as the spacing between each strip 31 on a single aid 10, assuming the strips 31 that define reference pattern 30 are placed parallel to Y₂. The medical scan image is a “slice” that is taken through the patient's body parallel to the X₂ direction of FIG. 9, perpendicular and transverse to the Y₂ direction of FIG. 10. In a resulting scan image, this alignment creates a series of reference marks 50 on the scan image that correspond to the positions of the strips 31 of reference pattern 30, and which are spaced nearly equal apart.

A common procedure that incurs body contour distortion is prostate treatment. For example, a cancer center may require an accurate body contour of CT slices starting from the L5 vertebra to 3 inches below the ischium. This length can vary between patients, depending on their height and body proportions, and can average 12 inches. Therefore, it would be safe to apply 14 inch strips of aid 10 on the pelvic area of an average height patient to cover the required body contour area. Other common procedures that incur body contour distortion are:

gynecologic/colorectal treatments, which may need a body contour from T12 to 6 inches below the ischium, or an average distance of 18 inches; and lung procedures, which may need a body contour from C6 to L5, or an average distance of 20 inches. Most patients would require between 3-5 strips of aid 10 per body side. One skilled in the art will understand these distances, suggested strip lengths and number of strips will vary based on the patient size, patient position, procedure type, treatment needs, treatment techniques and other treatment factors.

Turning now to FIGS. 11 and 12, and for purposes of illustration only, a strip of the aid 10, which includes three strips 31 of the radiopaque material is attached to a phantom 80, which is a schematic representation of a human body. FIG. 12 is a schematic cross sectional view along the line 12-12 of FIG. 11 and is a schematic representation of a scan slice 90A of the phantom 80 placed entirely within the sFOV. As may be seen, in the scan slice of FIG. 12 there are three reference marks 50 that appear as dots on the scan image and which correlate to the position of the strips 31 on the phantom 80.

FIG. 12 is an actual scan image, with reference number slice 90B, of the aid 10 attached to a cylindrical phantom 80 that had its left side partially placed within the 50-65 cm eFOV of a CT simulator such as an GE Optima CT580 RT. The right side of the image 90B contains the right side of the phantom 80 that remains within the sFOV. The corresponding CT image slice shows the right side of the phantom 80 being a perfect, or near perfect, semi-circle. The edges of right side outline represent the phantom's surface contour 88, which is analogous to a skin surface contour of a patient. The left side of the image 90B contains the left side of the phantom 80 and is within the CT simulator's 50-65 cm eFOV. The resulting image should ideally be a perfect semi-circle on the left side, which combined with the perfect semi-circle on the right side, would produce a perfect full circle that can be used to accurately measure SSD. However, due to employing extrapolation methods and image reconstruction algorithms of the CT simulator, the eFOV does not produce an accurate body contour. The resulting scan image 90B thus illustrates distortion 82 on the left side that is elongated instead of the perfect semi-circle that it should be. The body contour 84 that is generated by the automated treatment planning software estimates as the body contour may be seen as elongated on the left side of the phantom. The radiation therapy team may rely on this outline to calculate an inaccurate SSD.

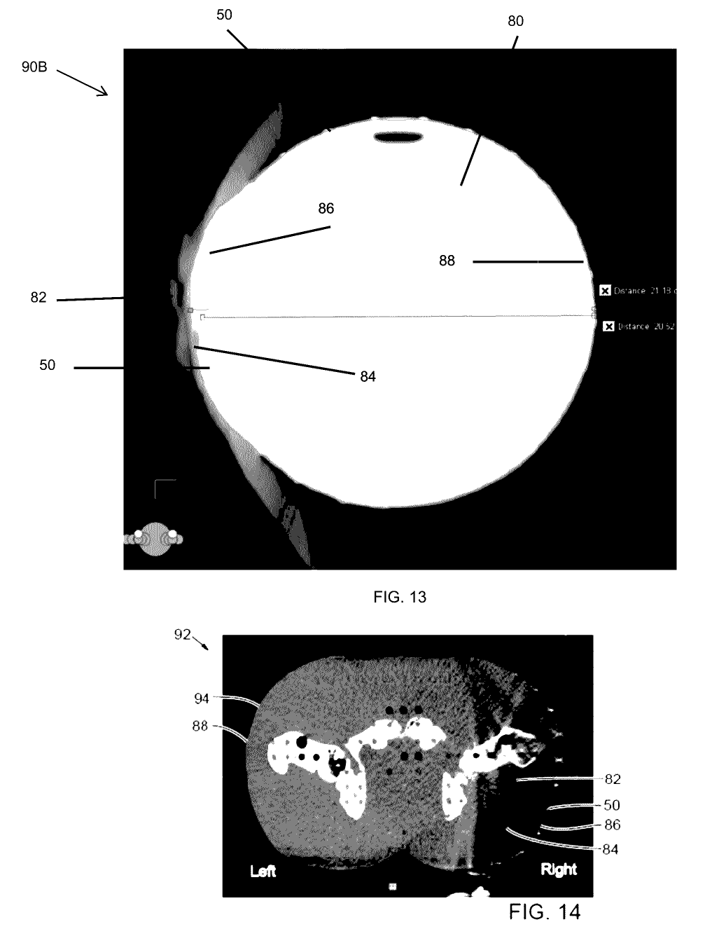

When the phantom is a perfect cylinder, it is obvious that the contour should be a perfect circle. However, a patient body contour is unique and not always symmetrical, and never a perfect cylinder. With continuing reference to FIG. 13, multiple strips of the aid 10 according to the invention were attached to portions of both the left and right side of the cylindrical phantom 80. The reference pattern 30 arising from the individual strips 31 appear as a series of visible reference marks 50 that appear as dots. The density of the radiopaque material used for strips 31, relative to the skin and flesh, is high enough that the CT simulator can accurately reconstruct the reference pattern 30 to represent the body contour without distortion or significant inaccuracy. The series of reference marks 50 can then be connected by the radiation therapy team using the appropriate treatment planning software. Preferably, the reference dots 50 are connected along the bottom of the dots—that is, the line that defines the connection between the reference dots is tangential to the lowermost edge of the dots—that is, the edge of the dots that is facing and closest to the interior of the patient's body. However, a good approximation may be made with any connection between the dots and the exact position of the dots will depend on factors such as the resolution. The radiation therapy team can create a manual body contour 86 that results in a near perfect circle. Connecting the reference marks 50 creates a body contour 86 through the elongated, distorted contour produced by the CT simulator. Connecting the reference marks 50 can be done directly underneath said marks as well, for a truer representation of the skin surface. However, one skilled in the art would understand that connecting through or above the dots would not have a significant impact on treatment planning.

FIG. 14 shows another phantom scan image in which the scan slice 92 contains strips of the aid 10 attached to a 33 cm pelvic phantom 94 that had its right side partially placed within the 50-78 cm HDFOV/eFOV of a Siemens Definition AS CT simulator. The edge of the right side of phantom 94 is placed at a 69 cm diameter and is within the scanner's eFOV. The left side of the phantom 94 remains within the sFOV. The corresponding CT image slice 92 shows the left side of the phantom 94 as an accurate outline of the pelvis, which is analogous to a skin surface contour of a patient. The right side of the image contains the right side of the phantom 94 and is within the CT simulator's 50-78 cm eFOV. The resulting image should ideally be a mirrored image of the left side, and an accurate full body contour would be used to measure SSD. However, the resulting scan image 92 illustrates a distorted contour 82 on the right side instead of the pelvic shape it should be. In comparison to FIG. 13, in which the distorted contour 82 does not need to be elongated and the distortion 82 is shortened and contracted from the true contour instead. However, in FIG. 14 the software body contour 84 can be seen as generally contracted on the right side of the phantom 94. The radiation therapy team may rely on this outline to calculate an inaccurate SSD. The aid 10 was attached to portions of the right side of the pelvic phantom 94 in FIG. 14 and the reference pattern 30 appears as a series of visible reference marks 50 that appear as dots on the scan image 92 (one reference mark 50 results from one strip 31). The series of reference marks 50 may then be connected by the radiation therapy team using the appropriate treatment planning software and create an accurate contour 86, which results in an accurate contour for calculation of the SSD.

The reference marks 50 in FIGS. 13 and 14 were connected using manual methods in the treatment planning software. This method and technique is not limited to creating manual contours by the radiation therapy team. The treatment planning software generally has the ability to create internal structure contours and external contours. The external contours are accurate for areas within the sFOV, but inaccurate for areas where distortion occurs. By improving the treatment planning software algorithms, the software could automatically connect the reference marks 50 without the user performing it manually, resulting in time savings.

Returning to FIG. 5, the aid 10 may be provided in a long strip form that is rolled for dispensing purposes. A rolled aid 10 such as that shown in FIG. 5 allows for increased user friendliness. A narrow rectangle, relative to its length, ideally conforms to the patient's skin contour compared to other shapes and designs. However, to cover an appropriate ROI, multiple strips are usually needed to increase the overall width that is being covered. Using pre-cut length strips has decreased dispensing efficiencies due to the user having to pick up and handle multiple strips. In addition, patient size differs and various lengths are needed. A rolled aid 10 solves both issues presented by pre-cut strips. A rolled aid 10 with perforations 28 allow the user to select the custom length that is needed for their patient. A pre-cut aid would result in waste from left over pieces that are not of appropriate length to use. A rolled aid 10 is also easier to handle and dispense compared to having to handle multiple individual strips. A rolled aid 10 can come in relatively long sizes, so users have less inventory to handle and require less frequency of re-stocking. For manufacturing purposes, the rolled aid 10 can be wound around an optional core 40. The protective release liner 22 may also be optional. The substrate 12, or at least the upper surface 20 thereof, could comprise the property that it is releasably attached to the lower surface 26 of the adhesive layer 14. As the underside 26 of the adhesive layer 14 comes in contact with the upper side 20 of the substrate, the upper side 20 of the substrate 12 acts as a protective liner equivalent in function to release liner 22 and thus protects the adhesive layer 14. The upper surface 20 of the substrate 12 and reference pattern 30 could also be coated with a material that has adhesive release properties to permit efficient unraveling of the aid 10.

FIG. 6 illustrates the rolled aid 10 inserted into a housing structure 100 for dispensing purposes. The housing structure 100 illustrated is preferably a cardboard, paper or plastic box. For illustrative purposes, the schematic shows the rolled aid 10 inserted in the housing structure 100. The housing structure may be clear or opaque. An opaque housing structure 100 would provide the means to print a label and instructions for a user. A transparent housing structure 100 could also accommodate printing, but the transparent property allows a user to conveniently see how much of the aid 10 remains before replacement is needed. The housing structure 100 can contain an opening with a flap 102 that holds a portion of the aid 10 out to create a tab 104 and prevent the aid 10 from rolling back into the housing structure 100. One skilled in the art would understand that the housing structure 100 is not limited to a box design and other dispensing systems may also be used. For example, the rolled aid 10 could be dispensed from a dispenser similar to 3M's Scotch® H180 Box Sealing Tape Dispenser or 3M's Scotch® DP300-RD Packaging Tape Hand Dispenser.

Turning now to FIG. 15, an alternative embodiment of the aid 10 is illustrated in which the aid 10 is in sheet form similar to the aid 10 shown in FIG. 1 except in which the reference pattern 30 is defined by strips 31 that are oriented at about 45 degrees with respect to X₁. Perforations 28 can also be included in both the X₁ and Y₁ directions so that smaller sizes of the aid 10 may be easily obtained. The size of the sheet can vary, but suggested ranges of the width W₁₀ can range from about 4.0 inches to about 8.0 inches and a length L₁₀ of about 6.0 to about 12.0 inches. As compared with the reference pattern 30 of FIG. 1, where the reference pattern 30 is defined by strips 31 that are parallel to X₁, the reference pattern 30 of FIG. 15 is defined by strips 31 that are oriented at an angle. Having a reference pattern 30 defined by angularly oriented strips 31 allows the user to orient the aid 10 so that either X₁ or Y₁ is parallel to Y₂. The angled reference pattern 30 allows the scan image slice to intersect the strips 31 so that the strips appear as dots on the scan, no matter if X₁ or Y₁ is parallel to Y₂. The angled reference pattern 30 does not require spacing between sheets/strips of the aid because the reference marks 50 that appear on the scan image will not have significant spacing gaps.

FIG. 16 shows a bird's eye and schematic view of a CT scan along the cross sectional slice 120 of the angled reference pattern 30, and the resulting reference marks 50 dots that would appear on the scan—the reference marks 50 are juxtaposed below the aids 10 in the drawing of FIG. 16. Multiple sheets of the aid 10—three in the case of FIG. 16, are placed close together and the dotted lines correspond to how each intersection of a strip 31 that is part of reference pattern 30 would appear on a scan image. By placing the aids 10 side by side, the maximum gap 124 between reference marks 50 is double the distance between each strip 31 of a reference pattern 30. It would not be recommended to space this embodiment further apart, as that could increase the gap size to unreasonable sizes.

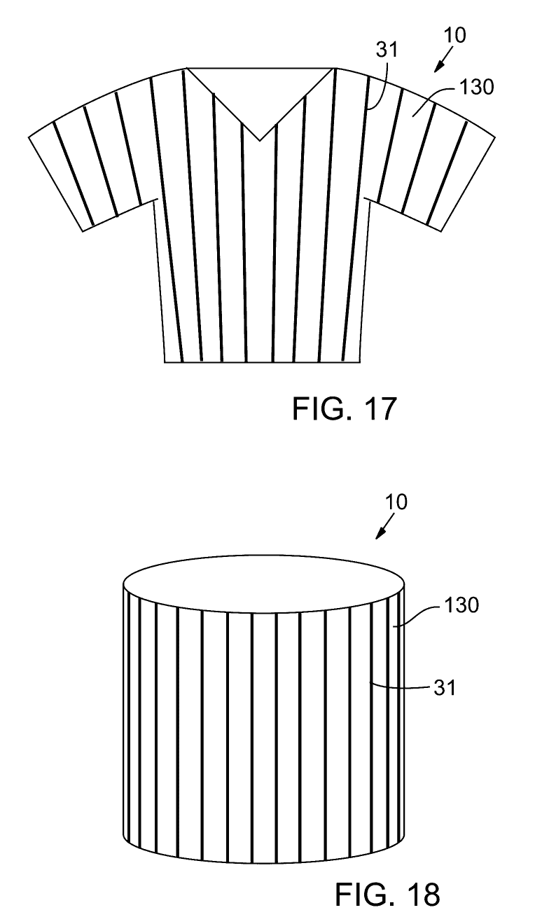

FIG. 17 shows an alternate embodiment of an aid 10 according to the invention, and wherein the aid is a wearable garment. Rather than a PO, or similar material and substrate that is adhered to the body, a wearable aid 10 is a fabric 130 such as a polyester-lycra material that is stretchy and form fitting so that it can accommodate and attach to different patient sizes and conform to the body contour. The reference pattern 30 defined by strips 31 is again defined by a series of radiopaque, parallel lines. The radiopaque lines, strips 31, could be screen printed on the fabric, metallic foil, or thin and flexible wires. The wearable aid 10 of FIG. 17 is similar in design to a tube top, but can also be a t-shirt to cover the shoulder and arms. This alternative embodiment works because it fulfills the four constraints of: (a) an at least partially radiopaque pattern, (b) conformability to the contours of the patient's body (i.e., skin), (c) attachment to the skin, and (d) covering a region of interest (ROI) that falls within the eFOV.

The schematic illustration of FIG. 18 represents the aid 10 shown in FIG. 17 (without arms, i.e. a tube top) on a phantom cylinder that represents a human body on which the wearable aid 10 is worn.

The method of using an aid 10 will now be detailed with specific reference to the aid 10 as shown in FIG. 1, and with reference to FIGS. 7 through 10. The patient 60 is first positioned on the medical imaging bed 62, usually a CT simulator 66. The patient 60 is positioned in the exact, or close to exact, position that will be replicated when the patient receives radiation therapy treatment. Thereafter, in step 2, the aid 10 is dispensed, for example from its housing structure 100 as shown in FIG. 6, and a section of aid 10 of the appropriate length based on the type of treatment and ROI 72 is torn off by the professional 68. In step 3, the adhesive layer 14 is exposed by separating the release liner 22 along the kiss cut 32. The flexible nature of the contour aid 10 allows the aid 10 to be placed substantially anywhere on the patient's body 60, and the aid 10 will conform to the contour of the patient's skin substantially without wrinkling or folding. Additionally, because the adhesive layer 14 attached to the skin surface is sufficiently distributed over the lower surface 18 of the substrate layer 12, the contour aid is securely attached to the skin surface to mitigate movement thereof or gapping between the aid and the skin or detachment from the skin during use. Those in the art will appreciate that, after the aid is attached, the contour of the patient's skin surface can change due to movement of the patient.

The aid 10 is placed on the patient such that the direction of the aid 10 is positioned so the strips 31 that define the reference pattern 30 intersect with the X₂ direction of the scanner bed 62. This orientation results in the width W₁₀ of the reference pattern 30 intersecting with the planes of the images or scans that are to be taken. This is generally an intersection between 45-90 degrees with respect to X₂. Thus, the reference pattern 30 will appear in the images or scans as a series of dots or dashes 50, which will represent the skin surface.

In step 4, the CT simulator scan is then taken and the aid 10 is removed from the patient 60 and discarded.

The CT simulator images are used for treatment planning purposes and in step 5, the radiotherapy professionals will choose the appropriate scanned cross-sectional image(s) or slice(s) and connect the reference marks 50 to create a body contour such as shown, for purpose of examples of FIGS. 13 and 14.

The method of use of the aid 10 according to the invention thus contemplates the following:
With the patient on the bed 62, identifying portions of the patient's body that extend into the eFOV—that is, portions 72 of the body that extend past the eFOV border 70 as shown in FIG. 8;
Adhering or otherwise attaching to the patient in body portion 72 the aid 10 according to the invention as described above;
Scanning the patient in the CT simulator pursuant to normal procedures;
On the scan image that results from the CT simulator scan, identifying the reference marks 50 that correspond to the locations of strips 31 from aid 10 through which the image slice was taken; and
Delineating the contour of the patient's body in the portions of the patient's body in the eFOV area by connecting the reference marks.

With the reference marks 50 thus connected, the technicians may determine accurately the SSD of the PTV. The body contour approximation defined by the connected reference marks provides an accurate representation of the body contour in the otherwise-distorted scan image in the eFOV area so that the SSD that is determined using the body contour approximation provides an accurate point from which the SSD may be determined.

As may be recognized by those of ordinary skill in the pertinent art based on the teachings herein, numerous changes and modifications may be made to the above-described and other embodiments without departing from the scope of the invention as defined in the appended claims. For example, the reference pattern may be made of other radiopaque materials, such as, for example, but not limited to, a thin and flexible copper wire. As another example, the liner may be made of any material capable of performing the functions of the liner as described herein. In addition, the contour aid may be used for other medical applications, as will be appreciated by those of ordinary skill in the art in view of the teachings herein. Accordingly, this detailed description of currently preferred embodiments is to be taken in an illustrative, as opposed to a limiting sense

While the present invention has been described in terms of preferred and illustrated embodiments, it will be appreciated by those of ordinary skill that the spirit and scope of the invention is not limited to those embodiments, but extend to the various modifications and equivalents as defined in the appended claims.

1. A method of creating a body contour delineation in a medical scan image in which the scan image encompasses a region of interest (ROI) on a subject being scanned and the ROI is at least partially in an extended field of view (eFOV) for the medical scan imaging apparatus, the method comprising the steps of:
a. identifying on the subject the ROI that extends into the eFOV;
b. providing a body contour delineation aid that comprises a radiopaque material and applying the body contour delineation aid onto the subject so that the radiopaque material extends over at least that portion of the ROI that is in the eFOV and such that the body contour delineation aid conforms to the subject;
c. performing a medical scan of the subject, the medical scan including the ROI;
d. generating a medical scan image from the medical scan so that the medical scan includes the body contour delineation aid in the ROI, wherein the radiopaque material in the eFOV appears on the medical image scan.

2. The method according to claim 1 including providing the body contour delineation aid in the form of an elongate strip to define a body contour delineation aid longitudinal axis and wherein the radiopaque material is defined by at least one strip of radiopaque material that extends parallel to the body contour delineation aid longitudinal axis.

3. The method according to claim 2 including the steps of:
a. positioning the subject on a bed associated with the medical scan imaging apparatus, wherein the bed defines an bed longitudinal axis; and
b. applying the body contour delineation aid so that the body contour delineation aid longitudinal axis is substantially parallel to the bed longitudinal axis.

4. The method according to claim 3 wherein the medical scan is taken along a plane that is transverse to the bed longitudinal axis.

5. The method according to claim 4 including the step of identifying in the eFOV on the medical scan image a reference mark that correlates to the location of the at least one strip of radiopaque material.

6. The method according to claim 5 in which the body contour delineation aid comprises three elongate strips of radiopaque material, each of the strips extending parallel to the body contour delineation aid longitudinal axis.

7. The method according to claim 5 including identifying in the eFOV on the medical scan image a reference mark that correlates to the location of each of the three strips of radiopaque material.

8. The method according to claim 7 including the step of connecting each reference mark to approximate body contour delineation in the eFOV.

9. The method according to claim 8 including determining a source-to-surface distance between a planning target volume located in a scan field of view (sFOV) in the medical scan image and the body contour delineation in the eFOV.

10. A method of delineating a patient's body contour in an extended field of view (eFOV) of a medical scan imaging apparatus, comprising the steps of:
a. providing a body contour delineation aid with a reference pattern thereon, the reference pattern defined by a material that is at least partially radiopaque;
b. identifying a portion of the patient's body that is in the eFOV of the medical scan imaging apparatus;
c. applying the body contour delineation aid to that portion of the patient's body that is in the eFOV;
d. performing a medical scan of the patient, the medical scan including scanning the portion of the patient's body that is in the eFOV to generate a medical scan image that comprises an image slice through the eFOV and the body contour delineation aid; and wherein the medical scan image has at least one reference mark corresponding to the reference pattern on the body contour delineation aid.

11. The method according to claim 10 in which the portion of the patient's body that is in the eFOV is irregular in surface topography and including the step of adhering the body contour delineation aid to the surface such that it conforms to the irregularities.

12. The method according to claim 11 in which the reference pattern is defined by plural parallel strips of the material that is at least partially radiopaque.

13. The method according to claim 12 wherein the medical scan image has plural reference marks, each reference mark in the plurality corresponding to one of the plural parallel strips.

14. The method according to claim 13 wherein the step of adhering the body contour delineation aid to the surface of the patient includes the step of orienting the body contour delineation aid so that the parallel strips extend transverse to the plane of the image slice.

15. The method according to claim 14 including the step of connecting each of the plural reference marks to define a body contour delineation for that portion of the patient's body that is in the eFOV.

16. The method according to claim 15 including determining a source-to-surface distance between a planning target volume located in a scan field of view (sFOV) in the medical scan image and the body contour delineation in the eFOV.

17. The method according to claim 16 including the step of generating the medical image scan with a CT simulator.

18. A body contour delineation aid for use with medical scan images, comprising:
a flexible and elongate substrate defining a longitudinal axis and having an upper surface and an opposite lower surface;
at least one strip of at least partially radiopaque material deposited on the upper surface;
an adhesive deposited on the lower surface.

19. The body contour delineation aid according to claim 18 including plural strips of at least partially radiopaque material deposited on the upper surface, each strip extending parallel to the longitudinal axis.

20. The body contour delineation aid according to claim 18 including plural strips of at least partially radiopaque material deposited on the upper surface, each strip parallel to the other strips and each strip extending at an angle relative to the longitudinal axis.
